(19) United States
(12) Patent Application Publication
(10) Pub. No.: US 2021/0093255 A1
(43) Pub. Date: Apr. 1, 2021

(54) Hearing Systems and Sensor Systems Including a Sensor Electrode and Methods for Manufacturing the Same

(71) Applicant: SONOVA AG, Staefa (CH)

(72) Inventors: Markus Mueller, Mannedorf (CH); Konstantin Silberzahn, Meilen (CH); Natasha Thumm, Wetzikon (CH)

(73) Assignee: SONOVA AG

(21) Appl. No.: 16/587,739

(22) Filed: Sep. 30, 2019

(51) Int. Cl. A61B 5/00; A61B 5/0478; A61B 5/04; H01R 12/70

(52) U.S. Cl. CPC A61B 5/6817; A61B 5/0478; H04R 2225/025; H01R 12/7076; A61B 5/04004

(57) ABSTRACT

An exemplary hearing system that is configured to assist a user in hearing includes an in-the-ear (“ITE”) component that comprises a shell having a contoured outer shape that is customized for the user to fit at least partially within an ear canal of the user, a faceplate configured to fit within an opening provided in the shell and face out of the ear canal of the user when the shell is inserted into the ear canal of the user, a sensor electrode that is provided on an outer surface of the shell, and a conductive path that extends along the outer surface of the shell from the sensor electrode to the faceplate and that conductively connects the sensor electrode with a conductive portion of the faceplate. Corresponding methods and systems are also disclosed.

### BACKGROUND INFORMATION

Hearing systems are used to improve the hearing capability and/or communication capability of users. Such hearing systems are configured to process a received input sound signal (e.g., ambient sound) and then provide the processed input sound signal to the user (e.g., through a hearing device such as a hearing aid). Hearing systems may use an in-the-ear (“ITE”) component to facilitate providing the processed input sound signal to the user. Such ITE components are configured to fit at least partially within an ear canal of the user.

In addition to being used to facilitate providing the processed input sound signal to the user, such ITE components may also include electrodes that may be configured to contact ear tissue within the ear canal of the user while the ITE component is worn by the user. Such electrodes may be used, for example, to detect electrical signals within tissue of the user. However, such electrodes may be difficult to manufacture due to space constraints within the ITE component. Moreover, to contact the ear tissue within the user's ear canal, the electrodes typically protrude from the outer surface of an ITE component. As such, the electrodes may make the ITE component itchy, painful, and/or generally uncomfortable for the user to wear.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary hearing system according to principles described herein.

FIG. 2 illustrates an exemplary ITE component of the hearing system shown in FIG. 1 according to principles described herein.

FIG. 3 illustrates an exemplary process for forming a sensor electrode and a conductive path on an outer surface of an ITE component according to principles described herein.

FIG. 4 illustrates an exemplary cross section of the ITE component shown in FIG. 2 that is taken along lines 4-4 in FIG. 2 according to principles described herein.

FIG. 5 illustrates an exemplary ITE component that includes a plurality of sensor electrodes according to principles described herein.

FIGS. 6 and 7 illustrate additional exemplary configurations of ITE components that may be implemented according to principles described herein.

FIG. 8 illustrates an exemplary sensor system according to principles described herein.

FIG. 9 illustrates an exemplary method for manufacturing a sensor electrode according to principles described herein.

### DETAILED DESCRIPTION

Hearing systems and sensor systems including a sensor electrode and methods for manufacturing the same are described herein. As will be described in more detail below, an exemplary hearing system configured to assist a user in hearing may include an ITE component that may comprise a shell having a contoured outer shape that is customized for the user to fit at least partially within an ear canal of the user, a faceplate configured to fit within an opening provided in the shell and face out of the ear canal of the user when the shell is inserted into the ear canal of the user, a sensor electrode that is provided on an outer surface of the shell, and a conductive path that extends along the outer surface of the shell from the sensor electrode to the faceplate and that conductively connects the sensor electrode with a conductive portion of the faceplate.

By providing a sensor electrode and a conductive path on an outer surface of a custom formed shell according to principles described herein, it is possible to provide an ITE component that is easier to manufacture and more comfortable for a user to wear than conventional ITE components that include electrodes. In addition, methods of manufacturing sensor electrodes such as those described herein may be more cost effective than methods of manufacturing conventional ITE components. Moreover, by manufacturing sensor electrodes and conductive paths according to principles described herein, it is possible to use space within ITE components more efficiently as compared to conventional ITE components. Other benefits of the hearing systems, sensor systems, and methods such as those described herein will be made apparent herein.

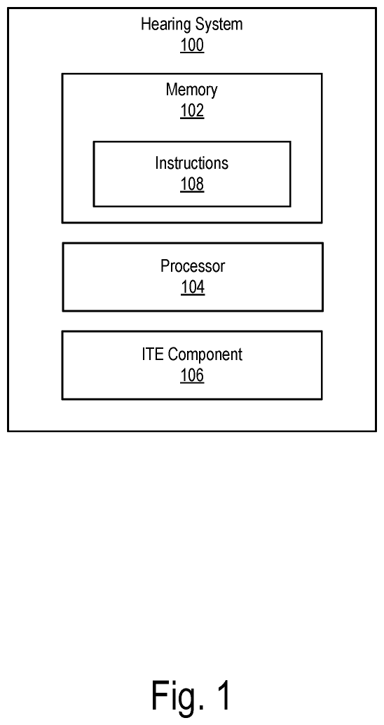

FIG. 1 illustrates an exemplary hearing system 100 that is configured to assist a user in hearing. As shown, hearing system 100 may include, without limitation, a memory 102, a processor 104, and an ITE component 106 selectively and communicatively coupled to one another. Memory 102 and processor 104 may each include or be implemented by hardware and/or software components (e.g., processors, memories, communication interfaces, instructions stored in memory for execution by the processors, etc.). In some examples, memory 102 and processor 104 may be housed within or form part of ITE component 106. In some examples, memory 102 and processor 104 may be located separately from ITE component 106 (e.g., in a behind-the-ear (“BTE”) component). In some alternative examples, memory 102 and processor 104 may be distributed between multiple devices (e.g., multiple hearing devices in a binaural hearing system) and/or multiple locations as may serve a particular implementation.

Memory 102 may maintain (e.g., store) executable data used by processor 104 to perform any of the operations associated with hearing system 100 described herein. For example, memory 102 may store instructions 108 that may be executed by processor 104 to perform any of the operations associated with hearing system 100 described herein. Instructions 108 may be implemented by any suitable application, software, code, and/or other executable data instance.

Memory 102 may also maintain any data received, generated, managed, used, and/or transmitted by processor 104. For example, memory 102 may maintain any suitable data associated with physiological attributes of a user that may be detected using one or more sensor electrodes such as those described herein. As used herein, a “physiological attribute” may refer to any characteristic that may be associated with the functioning of the body of the user of hearing system 100. For example, a physiological attribute may comprise a hydration level within the ear canal of the user, brain activity indicated in an electroencephalogram (“EEG”) measurement, a heartbeat attribute indicated in an electrocardiogram (“ECG”) measurement, and/or any other suitable physiological attribute. Memory 102 may maintain additional or alternative data in other implementations.

Processor 104 is configured to perform any suitable processing operation that may be associated with hearing system 100. For example, when hearing system 100 includes a hearing aid device, such processing operations may include monitoring ambient sound and/or representing sound to a user via an in-ear receiver. In examples where hearing system 100 is included as part of a cochlear implant system, such processing operations may include directing a cochlear implant to generate and apply electrical stimulation representative of one or more audio signals (e.g., one or more audio signals detected by a microphone, input by way of an auxiliary audio input port, etc.) to one or more stimulation sites associated with an auditory pathway (e.g., the auditory nerve) of a recipient. Processor 104 may be implemented by any suitable combination of hardware and software.

In addition, processor 104 is configured to perform any suitable processing operation associated with hearing system 100 using one or more sensor electrodes such as those described herein to detect one or more physiological attributes of a user.

ITE component 106 is configured to facilitate hearing system 100 (e.g., processor 104) detecting such physiological attributes of the user. To that end, ITE component 106 is configured to fit at least partially within an ear canal of a user and may include a shell, a faceplate, a sensor electrode, and a conductive path. Each of these elements will now be described in detail. Various configurations of ITE component 106 including these elements will then be described in connection with the figures.

The shell of ITE component 106 is configured to fit at least partially within an ear canal of the user and has a contoured outer shape that is customized for a user. The shell may correspond to any suitable type of shell that may be used in hearing system 100 and that has a contoured outer shape that is custom made for a particular user.

The shell may be formed of any suitable material as may serve a particular implementation. For example, the shell may be formed of any one or a combination polymers such as Nylon, acrylonitrile butadiene styrene (“ABS”), polycarbonate (“PC”), polyphenylene sulfide (“PPS”), polyetheretherketone (“PEEK”), liquid crystal polymer, acrylics, and/or any other suitable material.

The shell may be manufactured using any suitable manufacturing process as may serve a particular implementation. In certain examples, a three-dimensional (“3D”) printing process may be used to customize the shell to fit a particular user. By using a 3D printing process to manufacture a custom shell for a particular user, it is possible to reduce costs associated with manufacturing a custom shell as compared with other manufacturing methods such as injection molding. However, in certain alternative implementations, an injection molding process may be used to manufacture a custom shell to fit a particular user.

In examples where a shell is manufactured using a 3D printing process, 3D printable filaments that are amalgamated with an additive for laser direct structuring (“LDS”) may be used to form the shell. The additive used for LDS is configured to facilitate forming a sensor electrode and a conductive path on an outer surface of the shell during an LDS process. Any suitable additive may be used for LDS as may serve a particular implementation. For example, an additive used for LDS may include a metal oxide (e.g., tin oxide), an organometallic complex (e.g., a palladium/palladium-containing heavy metal complex), and/or any other suitable material. Exemplary operations associated with using an LDS process to form a sensor electrode and conductive path on an outer surface of a shell will be described herein.

In certain examples, the shell may be a molded interconnect device (“MID”) shell. MIDs are circuits that are directly integrated into the shape of a polymer component. An MID shell may be manufactured in any suitable manner. For example, an MID shell may be manufactured using an injection molding process in which any suitable material, such as those described herein, is injected into a mold to form the shape of the shell. The material injected into the mold may be amalgamated with an additive used for LDS, such as those described herein, to facilitate forming a sensor electrode and a conductive path on an outer surface of the shell during an LDS process.

The shell includes an opening that is configured to receive the faceplate to close the shell at a side oriented towards the exterior of the user's ear. The opening of the shell may have any suitable size and/or shape as may serve a particular implementation. In certain examples, the opening of the shell may have a standard size that is the same regardless of the particular shape of the ear canal of the user. In other implementations, the opening may have a custom shape that may be different for each particular user depending on the contoured outer shape of the shell.

The faceplate is configured to fit within the opening provided in the shell and face out of the ear canal of the user when the shell is inserted into the ear canal of the user. The faceplate may be formed of any suitable material. For example, the faceplate may be formed of the same material as the shell (e.g., Nylon, ABS, PC, PPS, PEEK, liquid crystal polymer, acrylics, etc.) or a different material.

The faceplate may be manufactured in any suitable manner. For example, in certain implementations, the faceplate may be 3D printed. In such examples, 3D printable filaments may be used to additively form the faceplate during a 3D printing process. In certain examples, the 3D printable filaments may be amalgamated with any suitable additive for LDS that may be used to form a conductive trace on a surface of the faceplate. Such a conductive trace may be configured to electrically connect the conductive path on the shell to electronics of the faceplate and/or electronics within the shell.

In certain alternative examples, the faceplate may be an injection molded faceplate formed of any suitable material for injection molding. For example, an injection molded faceplate may be formed of a thermoplastic material. Any suitable thermoplastic material may be used as may serve a particular implementation. For example, acrylics may be used to injection mold a faceplate in certain implementations. In certain examples, the material used to injection mold the faceplate may include an additive for LDS, such as those described herein. In such examples, the faceplate may be injection molded and then post processed as an MID in any suitable manner.

The faceplate may have any suitable configuration as may serve a particular implementation. For example, in certain implementations, the faceplate may have a standard shape that is configured to fit within a standard opening provided in the shell. Such a faceplate may be configured to fit within the standard opening in any suitable manner. For example, a faceplate with a standard shape may be clicked into the opening provided in the shell or may be glued into the opening. In such examples, the faceplate may be formed by cutting the faceplate to fit within the standard opening provided within the shell. Alternatively, the faceplate may be formed using an injection molding process.

In certain alternative implementations, the faceplate may be custom made to fit the contoured outer shape of the shell. For example, the faceplate may be cut in any suitable manner to fit the contoured outer shape of the shell. Alternatively, a 3D printing process may be used to manufacture the faceplate.

In certain examples, the faceplate may include one or more elements that facilitate operation and/or manual adjustment of hearing system 100. For example, in certain implementations, the faceplate may include a push button (e.g., that may be used to change hearing aid programs), a volume adjustment dial, a battery door usable to access a battery housed within the shell, and/or any other suitable element. In certain alternative implementations, the faceplate may not include any elements that facilitate manual adjustment of system 100. Rather, the faceplate may simply cover the opening in the shell and facilitate communicatively connecting the sensor electrode to circuitry within the shell or within another component such as a BTE component.

Additionally or alternatively, the faceplate may include one or more microphones, a vent to aid in the reduction of occlusion, a removal handle, and/or any other suitable feature.

At least a portion of the faceplate is configured to be conductive such that the conductive path connects the sensor electrode to a conductive portion of the faceplate. The conductive portion of the faceplate conductively connects the faceplate to any suitable circuitry or electronics that may be provided within the shell and/or outside of the shell (e.g., in a BTE component). The conductive portion of the faceplate may be formed in any suitable manner. For example, at least a portion of the faceplate may be provided with any suitable conductive particles.

Alternatively, a conductive trace may be formed on a portion of the faceplate in any suitable manner. For example, a conductive trace on the faceplate may be formed through an LDS process similar to that described herein with respect to the conductive path and sensor electrode on the outer surface of the shell. Such a conductive trace may be formed of any suitable metal or combination of metals. For example, the conductive trace may be formed of copper, nickel, gold, and/or any other suitable metal or combination of metals. In certain examples, the conductive portion of the faceplate may electrically connect to a printed circuit board (“PCB”) provided on the faceplate.

In certain examples, the faceplate may include additional or alternative components to facilitate connecting electronics of the faceplate and/or electronics within the shell to the conductive path and the sensor electrode on the outer surface of the shell. For example, the faceplate may include inlays (e.g., metal rods, frames, etc.) or a PCB that may be directly integrated into the faceplate. Such features may be integrated into the faceplate in any suitable manner. For example, such features may be integrated into the faceplate during an injection molding process and/or an overmolding process. Alternatively, in examples where the faceplate has a standard shape, such features may be attached to the faceplate (e.g., by gluing) after the faceplate is formed.

In certain examples, the faceplate may include a rim configured to overlap the outer surface of the shell when the faceplate is inserted within the opening in the shell. In such examples, the conductive portion of the faceplate may be included on the rim such that, when the faceplate is inserted into the opening in the shell, the conductive portion electrically connects circuitry of hearing system 100 (e.g., within the shell) to the sensor electrode by way of the conductive path.

The sensor electrode is configured to be provided on an outer surface of the shell and is manufactured so as to follow the contoured outer shape of the shell. The sensor electrode may be used to detect any suitable information associated with the user. For example, the sensor electrode may be used to detect one or more physiological attributes of the user, such as those described herein.

The sensor electrode and the conductive path may be manufactured in any suitable manner. For example, the sensor electrode and conductive path may be manufactured through an LDS process, such as will be described herein. Alternatively, the sensor electrode and the conductive path may be formed through either an aerosol jet printing process or a ProtoPaint LDS process in certain implementations.

The sensor electrode and the conductive path may be formed of any suitable metal or combination of metals. For example, the sensor electrode may be formed of copper, nickel, gold, and/or any other suitable metal or combination of metals.

Any suitable number of sensor electrodes may be provided on the outer surface of the shell as may serve a particular implementation. For example, in certain implementations, only one sensor electrode may be provided on the outer surface of the shell. Alternatively, a plurality of sensor electrodes may be arranged in any suitable manner on the outer surface of the shell.

The sensor electrode may have any suitable, configuration, size, and/or shape as may serve a particular implementation. For example, the sensor electrode may be shaped as a square, a rectangle, a circle, etc. In certain examples, the sensor electrode may have a specific layout configured to facilitate detecting a particular type of physiological attribute of the user. For example, the sensor electrode may include a plurality of parallel electrode strips configured to create an electromagnetic field that may be used to determine skin capacitance of the user.

The sensor electrode may be positioned in any suitable location on the outer surface of the shell such that the sensor electrode is in direct contact with tissue within the ear canal when ITE component 106 is inserted within the ear canal. In certain examples, the sensor electrode may be located within a sealing zone associated with ITE component 106. As used herein, a “sealing zone” refers to a region around an outer circumference of the shell that has the acoustic function to seal the ear canal when ITE component 106 is worn by the user. It is understood that the outer surface of the shell that is in the sealing zone is in direct contact with the ear tissue in the ear canal when the shell is provided within the ear canal. As such, the sensor electrode may be positioned in any suitable location within the sealing zone to ensure that the sensor electrode is in direct contact with ear tissue of the user while ITE 106 component is worn by the user.

The conductive path is configured to electrically connect the sensor electrode to the faceplate. The conductive path is provided on the outer surface of the shell and, similar to the sensor electrode, is configured to follow the curvature of the outer surface of the shell. The conductive path may have any suitable thickness on the outer surface and/or may have any suitable length as may serve a particular implementation. In addition, the conductive path may take any suitable route from the sensor electrode to the faceplate. In certain examples, the conductive path may be covered with a thin protective coating made of any suitable material to protect and/or insulate the conductive path.

The electrical connection between the conductive path and the conductive portion of the faceplate may be facilitated in any suitable manner. For example, in certain implementations, the faceplate may overlap the shell when inserted within the opening in the shell such that the conductive portion of the faceplate is in direct contact with the conductive path on the outer surface of the shell. Additionally or alternatively, the electrical connection between the conductive path and the faceplate may be facilitated by using conductive glue, soldering, spring contacts, bolts with press-fit, and/or in any other suitable manner. In so doing, it may be possible to easily electrically connect the conductive path to circuitry on the faceplate, in the shell, and/or outside of the shell by way of the faceplate.

ITE component 106 may have any other suitable components as may serve a particular implementation. For example, in certain implementations, ITE component 106 may include a microphone configured to detect an audio signal. Additionally or alternatively, ITE component 106 may include a receiver (e.g., a speaker configured to deliver an audio signal to the user.

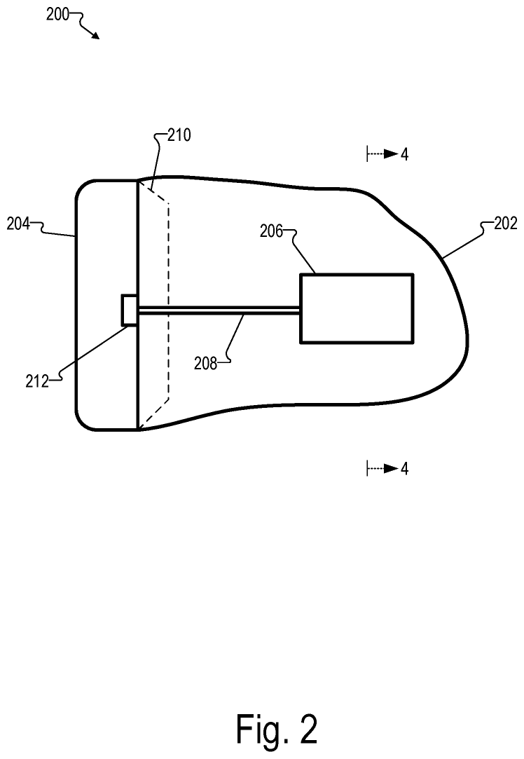

FIG. 2 shows an exemplary configuration 200 of ITE component 106. As shown in FIG. 2, ITE component 106 includes a shell 202, a faceplate 204, a sensor electrode 206, and a conductive path 208, each of which may be implemented as described above. As shown in FIG. 2, shell 202 has a contoured outer shape that is customized for a user to fit at least partially within an ear canal of the user. Faceplate 204 is configured to fit within an opening 210 of shell 202. In the example shown in FIG. 2, opening 210 of shell 202 is indicated by dashed lines and opens to the left to receive faceplate 204 and close shell 202 at a side oriented towards the exterior of the user's ear.

Sensor electrode 206 is provided on an outer surface of shell 202 at a position such that, when inserted within the ear canal, sensor electrode 206 may be in direct contact with ear tissue of the user within the ear canal.

Conductive path 208 conductively connects sensor electrode 206 to faceplate 204. As shown in FIG. 2, conductive path 208 extends along the outer surface of shell 202 from sensor electrode 206 to a conductive portion 212 of faceplate 204.

In the example shown in FIG. 2, conductive portion 212 is depicted as having a rectangular shape for illustrative purposes. However, it is understood that conductive portion 212 may be configured in any suitable manner, such as described herein. For example, conduct portion 212 may have any suitable shape and/or size as may serve a particular implementation. In addition, in certain examples, conductive portion 212 may be visible, as shown in FIG. 2. Alternatively, conductive portion 212 may not be visually discernable from other portions of faceplate 204 in certain implementations.

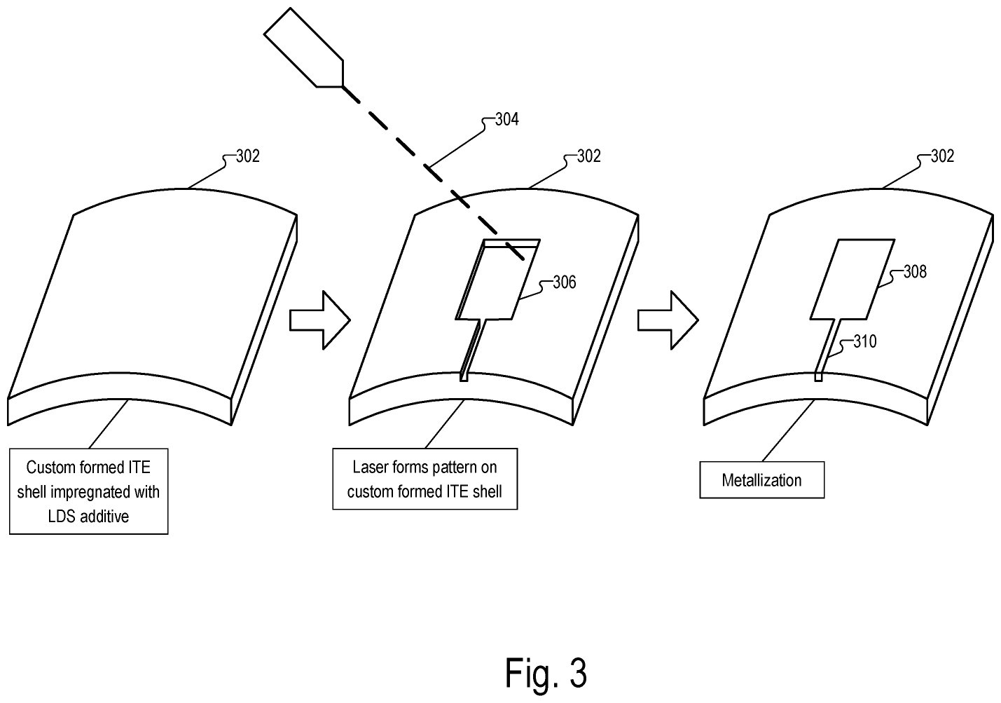

Sensor electrode 206 and conductive path 208 are formed such that they follow the contoured outer surface of shell 202. This is beneficial in that it results in ITE component 106 being more comfortable to wear than conventional ITE components. To manufacture sensor electrode 206 and conductive path 208 so that they have such characteristics, an LDS process may be used in certain implementations. Various operations that may performed during an LDS process will now be described with reference to FIG. 3.

As shown in FIG. 3, the leftmost image corresponds to a portion 302 of the outer surface of a custom formed ITE component shell (e.g., shell 202 of ITE component 106) that is amalgamated with an LDS additive (e.g., an organometallic additive). Although only portion 302 is shown in FIG. 3, it is understood that the LDS process shown in FIG. 3 would be performed on the whole custom formed ITE component shell (e.g., shell 202).

In the middle image of the LDS process shown in FIG. 3, an activation process is performed in which a computer-controlled laser beam 304 travels over portion 302 to expose a pattern 306 for the sensor electrode and the conductive path and activate the LDS additive. This activation process causes a physical-chemical reaction that produces metallic nuclei to which metal will attach during a metallization process. In addition to activating the LDS additive, laser beam 304 may form a microrough surface on which the metal can attach during the metallization process.

In the rightmost image shown in FIG. 3, a metallization process has been performed to form a sensor electrode 308 and a conductive path 310. Such a metallization process may be performed in any suitable manner. For example, the metallization process may include an additive conductor build-up performed in electroless copper baths. In certain examples, a plurality of different metals may be sequentially added to form sensor electrode 308 and conductive path 310. For example, electroless copper plating may be used to form a first metal layer formed of copper on the pattern. Afterwards, electroless nickel plating may be used to form a second metal layer of nickel on the second metal layer formed of copper. In certain examples, an electroless gold plating may also be used to form a third metal layer formed of gold on the second metal layer formed of nickel. Other types and/or combinations of metals may be built-up during the metallization process in other implementations.

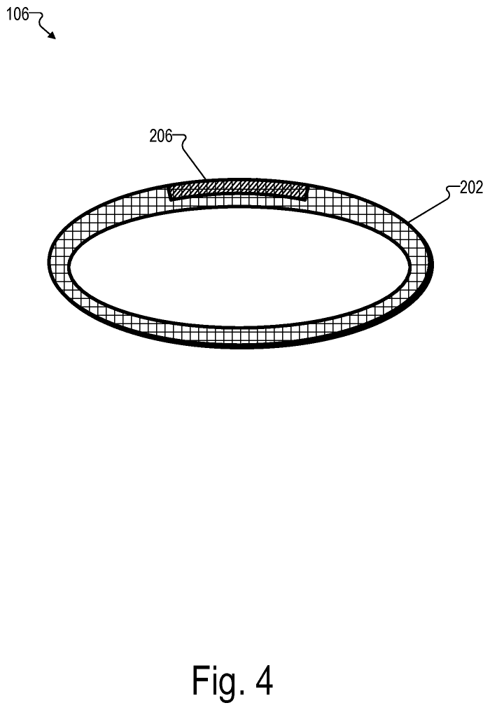

By using the LDS process described above, it is possible to manufacture sensor electrodes and conductive paths that follow the contoured outer shape of a shell and, as a result, are more comfortable when in contact with ear tissue of a user within the ear canal. To illustrate, FIG. 4 shows a cross-section of shell 202 shown in FIG. 2 that is taken along lines 4-4 in FIG. 2. As shown in FIG. 4, sensor electrode 206 has a contour that follows the contoured shape of the outer surface of shell 202 and is flush with the outer surface. With such a configuration, pressure from sensor electrode 206 is distributed evenly when shell 202 is worn by the user. As such, it is possible to mitigate harm and/or discomfort to the user.

In FIG. 4, the cross-sectional thickness of sensor electrode 206 is exaggerated for illustrative purposes. It is understood that the cross-sectional thickness of sensor electrode 206 may be relatively much less than the cross-sectional thickness of the wall of shell 202. For example, sensor electrode 202 may have a cross-sectional thickness on the order of a few microns whereas the wall of shell 202 may have a cross-sectional thickness on the order of a few millimeters. In addition, for simplicity, FIG. 4 shows the space within shell 202 as being empty. However, it is understood that the interior space may include any suitable circuitry, electronics, and/or other structures in certain implementations.

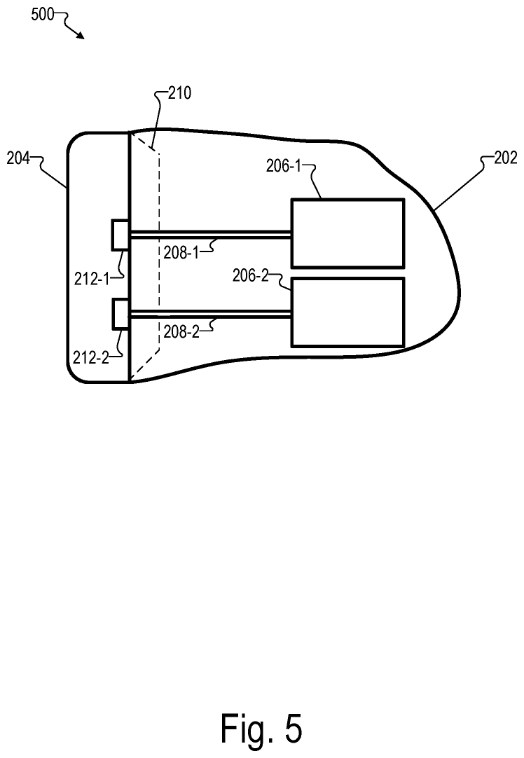

In certain examples, an additional sensor electrode may be provided on an outer surface of a shell. Such an additional sensor electrode may be electrically connected to a faceplate by way of an additional conductive path that extends along the outer surface of the shell from the additional sensor electrode to the faceplate. To illustrate, FIG. 5 shows an exemplary configuration 500 of ITE component 106 that includes two sensor electrodes 206 (e.g., sensor electrodes 206-1 and 206-2) that are each respectively electrically connected to conductive portions 210 (e.g., conductive portions 210-1 and 210-2) on faceplate 204 by way of conductive paths 208 (e.g., conductive paths 208-1 and 208-2). Although FIG. 5 shows sensor electrode 206-1 as being adjacent to sensor electrode 206-2, it is understood that sensor electrodes 206 could be arranged in any suitable manner with respect to one another in other implementations. For example, sensor electrode 206-1 may be provided on one side of shell 202 and sensor electrode 206-2 may be provided on an opposite side of shell 202 in certain implementations.

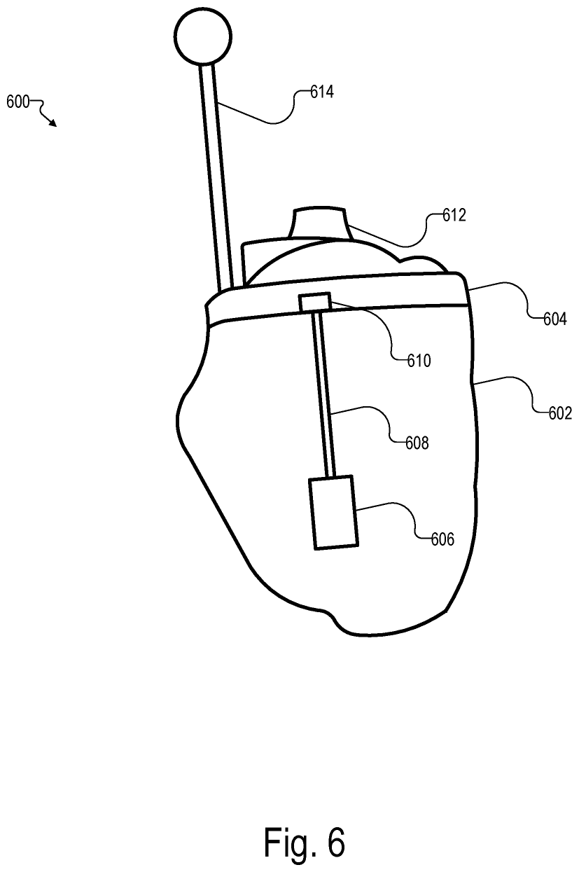

FIG. 6 shows an exemplary configuration 600 of ITE component 106 that may be provided in certain implementations when ITE component 106 corresponds to a hearing aid device. As shown in FIG. 6, configuration 600 includes a shell 602, a faceplate 604, a sensor electrode 606, and a conductive path 608 that electrically connects sensor electrode 606 to a conductive portion 610 of faceplate 604. In addition, configuration 600 includes a push button 612 and a removal handle 614. Push button 612 may be used, for example, to change a hearing program and/or a volume level. Removal handle 614 may assist in removing ITE component 106 from the ear canal of the user. Further, in configuration 600, processor 104 and/or other electronics may be housed within shell 602. Furthermore, in configuration 600, faceplate 604 of is customized to the shape of an opening provided in shell 602. As such, faceplate 604 is custom made in any suitable manner, such as described herein, for a particular recipient.

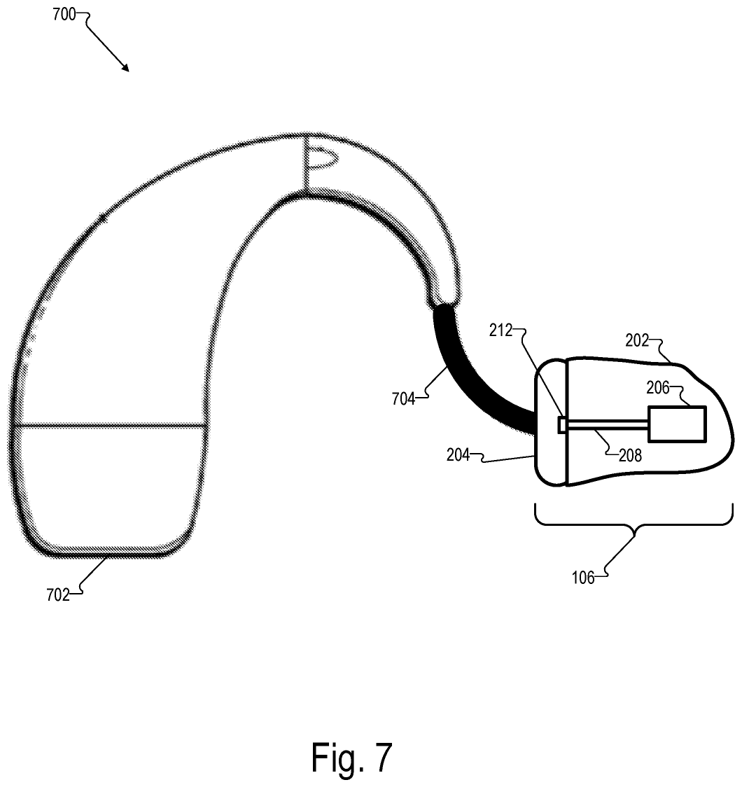

In certain examples, hearing system 100 may further comprise a BTE component that is communicatively coupled to ITE component 106. Such a BTE component is configured to be worn behind an ear of a user and may include circuitry (e.g., a processor similar to processor 104) configured to control operation of a sensor electrode. In such examples, ITE component 106 may further include a receiver (e.g., a speaker) configured to acoustically deliver an audio signal to the user as directed by the BTE component. The BTE component may be communicatively coupled to ITE component 106 in any suitable manner. For example, FIG. 7 illustrates an exemplary configuration 700 in which ITE component 106 is connected to a BTE component 702 by way of a connector portion 704, which may be implemented by any suitable wired connection. In the example shown in FIG. 7, sensor electrode 206 may be electrically connected to circuitry within BTE component 702 by way of conductive path 208, conductive portion 212, and, for example, one or more wires (not shown) that extend within connector portion 704 from faceplate 204 to BTE component 702.

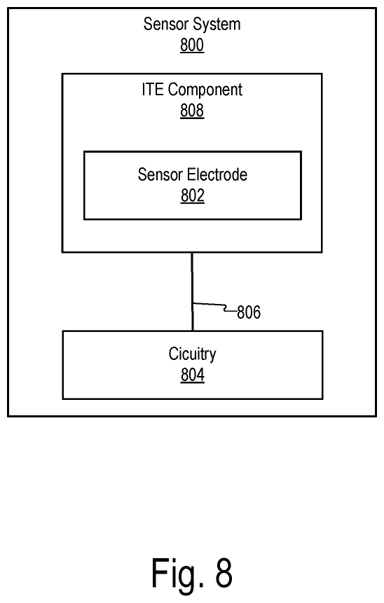

FIG. 8 illustrates an exemplary sensor system 800 that may be implemented in certain examples according to principles described herein. As shown in FIG. 8, sensor system 800 includes a sensor electrode 802, circuitry 804, and a conductive path 806. Sensor electrode 802 is configured to be provided on an outer surface of a shell of an ITE component 808 in any suitable manner, such as described herein.

Circuitry 804 may be provided in any suitable location as may serve a particular implementation. For example, circuitry 804 may be provided within a shell of ITE component 808 in certain examples. In such examples, the conductive portion (e.g., conductive portion 212) of the faceplate (e.g., faceplate 204) may be conductively connected to the circuitry housed within the shell. Alternatively, circuitry 804 may be housed within a BTE component (e.g., BTE component 702) that is communicatively coupled to the ITE component. In such examples, the ITE component may further comprise a receiver configured to acoustically deliver an audio signal to the user as directed by the BTE component.

In certain examples, a sensor electrode (e.g., sensor electrode 206) may be conductively connected to circuitry within a shell (e.g., shell 202) by way of a through hole provided in the shell instead of by way of a conductive path that is provided on an outer surface of the shell and that connects to a faceplate (e.g., faceplate 204). In such examples, one or more wires (e.g., litzwires) may be provided through the through hole so as to connect the sensor electrode to electronic components provided within the shell. The one or more wires may be connected in the through hole in any suitable manner. For example, the one or more wires may be connected in the through hole by way of conductive glue, soldering, spring contacts, bolts with a press-fit, etc. In certain examples, the sensor electrode may overlap the through hole provided in the shell. For example, the sensor electrode may be centered over the through hole. In such examples, the sensor electrode may be fixed within the through hole by using glue, any suitable mechanical retention mechanism, and/or a spring.

In certain alternative examples, the through hole may be offset from the sensor electrode on the outer surface of the shell. In such examples, the sensor electrode may be communicatively coupled to one or more wires in the through hole by way of a conductive path that extends along the outer surface of the shell from the sensor electrode to the through hole. When the sensor electrode is offset from the through hole, the sensor electrode and/or conductive path that extends to the through hole may be formed in any suitable manner (e.g., by an LDS process), such as described herein.

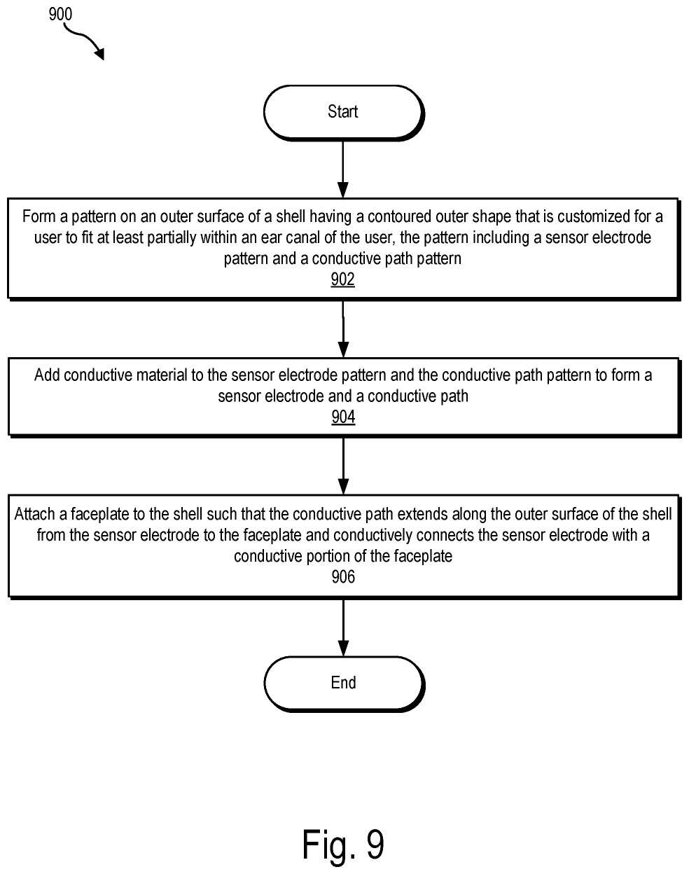

FIG. 9 illustrates an exemplary method for manufacturing a sensor electrode according to principles described herein. While FIG. 9 illustrates exemplary operations according to one embodiment, other embodiments may omit, add to, reorder, and/or modify any of the operations shown in FIG. 9.

In operation 902, a pattern is formed on an outer surface of a shell that has a contoured outer shape that is customized for a user to fit at least partially within an ear canal of the user. The pattern may include a sensor electrode pattern and a conductive path pattern. As described herein, the pattern may be formed by using an LDS process in which a computer-controlled laser forms the pattern and activates an LDS additive included in the shell. Operation 902 may be performed in any of the ways described herein.

In operation 904, conductive material is added to the sensor electrode pattern and the conductive path pattern to form a sensor electrode and a conductive path. As described herein, the conductive material may be added through an electroless plating operation in which one or more metals are added to the sensor electrode pattern and the conductive path pattern. Operation 904 may be performed in any of the ways described herein.

In operation 906, a faceplate is attached to the shell such that the conductive path extends along the outer surface of the shell from the sensor electrode to the faceplate and conductively connects the sensor electrode with a conductive portion of the faceplate. In certain examples, the attaching of the faceplate to the shell may include fitting the faceplate within a custom opening provided in the shell. Alternatively, the attaching of the faceplate to the shell may include fitting a standard faceplate within a standard opening provided in the shell. Operation 906 may be performed in any of the ways described herein.

In certain examples, the method illustrated in FIG. 9 may further include 3D printing the shell such that the shell is customized for the user to fit at least partially within the ear canal of the user.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A hearing system configured to assist a user in hearing, the hearing system comprising:
an in-the-ear (ITE) component comprising:
a shell having a contoured outer shape that is customized for the user to fit at least partially within an ear canal of the user;
a faceplate configured to fit within an opening provided in the shell and face out of the ear canal of the user when the shell is inserted into the ear canal of the user;
a sensor electrode that is provided on an outer surface of the shell; and
a conductive path that extends along the outer surface of the shell from the sensor electrode to the faceplate and that conductively connects the sensor electrode with a conductive portion of the faceplate.

2. The hearing system of claim 1, wherein the shell is a three-dimensional printed shell that is custom printed for the user to fit at least partially within the ear canal of the user.

3. The hearing system of claim 2, wherein:
the shell is formed of a material that includes an additive for laser direct structuring (LDS); and
the sensor electrode and the conductive path are patterned on the outer surface of the shell by an LDS process that activates the additive for LDS.

4. The hearing system of claim 1, wherein the faceplate is an injection molded faceplate that is formed of a thermoplastic material that includes an additive for laser direct structuring (LDS).

5. The hearing system of claim 1, wherein the faceplate is a three-dimensional printed faceplate that is custom printed for the user to fit the contoured outer shape of the shell.

6. The hearing system of claim 1, wherein the conductive portion of the faceplate is conductively connected to circuitry housed within the shell.

7. The hearing system of claim 1, further comprising a behind-the-ear (BTE) component that is communicatively coupled to the ITE component and that includes circuitry configured to control operation of the sensor electrode,
wherein the ITE component further comprises a receiver configured to acoustically deliver an audio signal to the user as directed by the BTE component.

8. The hearing system of claim 1, wherein the ITE component further comprises:
a microphone configured to detect an audio signal; and
a receiver configured to deliver the audio signal to the user.

9. The hearing system of claim 1, wherein the sensor electrode is configured to detect a physiological attribute of the user.

10. The hearing system of claim 1, wherein the conductive path is conductively connected to the conductive portion of the faceplate by at least one of conductive glue, soldering, spring contacts, and bolts with press-fit.

11. The hearing system of claim 1, wherein the ITE component further comprises:
an additional sensor electrode provided on the outer surface of the shell; and
an additional conductive path that extends along the outer surface of the shell from the additional sensor electrode to the faceplate and that conductively connects the additional sensor electrode with an additional conductive portion of the faceplate.

12. A hearing system configured to assist a user in hearing, the hearing system comprising:
an in-the-ear (ITE) component comprising:
a shell having a contoured outer shape that is customized for the user to fit at least partially within an ear canal of the user;
a faceplate configured to fit within an opening provided in the shell and face out of the ear canal of the user when the shell is inserted into the ear canal of the user; and
a sensor electrode that is provided on a surface of the shell,
wherein the shell is formed of a material that includes an additive for laser direct structuring (LDS).

13. A sensor system for use with an in-the-ear (ITE) component of a hearing system configured to assist a user in hearing, the sensor system comprising:
a sensor electrode provided on an outer surface of a shell of the ITE component, the shell having a contoured outer shape that is customized for the user to fit at least partially within an ear canal of the user;
circuitry configured to use the sensor electrode to detect a physiological attribute of the user; and
a conductive path that extends along the outer surface of the shell from the sensor electrode to a faceplate of the ITE component and that conductively connects the sensor electrode to a conductive portion on the faceplate.

14. The sensor system of claim 13, wherein:
the circuitry is housed within the shell of the ITE component; and
the conductive portion of the faceplate is conductively connected to the circuitry housed within the shell.

15. The sensor system of claim 13, wherein:
the circuitry is housed within a behind-the-ear (BTE) component that is communicatively coupled to the ITE component; and
the ITE component further comprises a receiver configured to acoustically deliver an audio signal to the user as directed by the BTE component.

16. The sensor system of claim 13, wherein:
the shell is formed of a material that comprises an additive for laser direct structuring (LDS); and
the sensor electrode and the conductive path are patterned on the outer surface of the shell by an LDS process that activates the additive for LDS.

17. The sensor system of claim 13, wherein physiological attribute comprises at least one of a hydration level within the ear canal of the user, brain activity indicated in an electroencephalogram (EEG) measurement, and a heartbeat attribute indicated in an electrocardiogram (ECG) measurement.

18. A method of manufacturing an in-the-ear (ITE) component of a hearing system configured to assist a user in hearing, comprising:
forming a pattern on an outer surface of a shell having a contoured outer shape that is customized for the user to fit at least partially within an ear canal of the user, the pattern including a sensor electrode pattern and a conductive path pattern;
adding conductive material to the sensor electrode pattern and the conductive path pattern to form a sensor electrode and a conductive path; and
attaching a faceplate to the shell such that the conductive path extends along the outer surface of the shell from the sensor electrode to the faceplate and conductively connects the sensor electrode with a conductive portion of the faceplate.

19. The method of claim 18, wherein the forming of the pattern on the outer surface of the shell includes using a laser direct structuring (LDS) process to form the pattern and activate an additive for LDS included within the shell.

20. The method of claim 18, further comprising three-dimensionally printing the shell such that the shell is customized for the user to fit at least partially within the ear canal of the user.
